SEQUENCE LISTING (8 sequences)

SEQ ID NO: 1 - DNA, 1652 bp, human
aaaaacatgatgagaagtctataaaaattgtgtgctaccaaagatctgtcttatttggcagctgctgcctcacccacagcttttgatatctaggaggactcttctctcccaaactacctgtcaccatggcccaccgatttccagccctcacccaggagcagaagaaggagctctcagaaattgcccagagcattgttgccaatggaaaggggatcctggctgcagatgaatctgtaggtaccatggggaaccgcctgcagaggatcaaggtggaaaacactgaagagaaccgccggcagttccgagaaatcctcttctctgtggacagttccatcaaccagagcatcgggggtgtgatccttttccacgagaccctctaccagaaggacagccagggaaagctgttcagaaacatcctcaaggaaaaggggatcgtggtgggaatcaagttagaccaaggaggtgctcctcttgcaggaacaaacaaagaaaccaccattcaagggcttgatggcctctcagagcgctgtgctcagtacaagaaagatggtgttgactttgggaagtggcgtgctgtgctgaggattgccgaccagtgtccatccagcctcgctatccaggaaaacgccaacgccctggctcgctacgccagcatctgtcagcagaatggactggtacctattgttgaaccagaggtaattcctgatggagaccatgacctggaacactgccagtatgttactgagaaggtcctggctgctgtctacaaggccctgaatgaccatcatgtttacctggagggcaccctgctaaagcccaacatggtgactgctggacatgcctgcaccaagaagtatactccagaacaagtagctatggccaccgtaacagctctccaccgtactgttcctgcagctgttcctggcatctgctttttgtctggtggcatgagtgaagaggatgccactctcaacctcaatgctatcaacctttgccctctaccaaagccctggaaactaagtttctcttatggacgggccctgcaggccagtgcactggctgcctggggtggcaaggctgcaaacaaggaggcaacccaggaggcttttatgaagcgggccatggctaactgccaggcggccaaaggacagtatgttcacacgggttcttctggggctgcttccacccagtcgctcttcacagcctgctatacctactagggtccaatgcccgccagcctagctccagtgcttctagtaggagggctgaaagggagcaacttttcctctaatcctggaaattcgacacaattagatttgaactgctggaaatacaacacatgttaaatcttaagtacaagggggaaaaaataaatcagttattgaaacataaaaatgaataccaaggacctgatcaaatttcacacagcagtttccttgcaacactttcagctccccatgctccagaatacccacccaagaaaataataggctttaaaacaatatcggctcctcatccaaagaacaactgctgattgaaacacctcattagctgagtgtagagaagtgcatcttatgaaacagtcttagcagtggtaggttgggaaggagatagctgcaaccaaaaaagaaataaatattctataaaccttc

SEQ ID NO: 2 - DNA, 5215 bp, human
aagcaaccttaaaatgactgcaccctcccagatttcttttacattaactaaaaagtcttatcacacaatctcataaaatttatgtaatttcatttaattttagccacaaatcatcaaaatgacgaggattttgacagctttcaaagtggtgaggacactgaagactggttttggctttaccaatgtgactgcacaccaaaaatggaaattttcaagacctggcatcaggctcctttctgtcaaggcacagacagcacacattgtcctggaagatggaactaagatgaaaggttactcctttggccatccatcctctgttgctggtgaagtggtttttaatactggcctgggagggtacccagaagctattactgaccctgcctacaaaggacagattctcacaatggccaaccctattattgggaatggtggagctcctgatactacttctctggatgaactgggacttagcaaatatttggagtctaatggaatcaaggtttcaggtttgctggtgctggattatagtaaagactacaaccactggctggctaccaagagtttagggcaatggctacaggaagaaaaggttcctgcaatttatggagtggacacaagaatgctgactaaaataattcgggataagggtaccatgcttgggaagattgaatttgaaggtcagcctgtggattttgtggatccaaataaacagaatttgattgctgaggtttcaaccaaggatgtcaaagtgtacggcaaaggaaaccccacaaaagtggtagctgtagactgtgggattaaaaacaatgtaatccgcctgctagtaaagcgaggagctgaagtgcacttagttccctggaaccatgatttcaccaagatggagtatgatgggattttgatcgcgggaggaccggggaacccagctcttgcagaaccactaattcagaatgttcagaagattttggagagtgatcgcaaggagccattgtttggaatcagtacaggaaacttaataacaggattggctgctggtgccaaaacctacaagatgtccatggccaacagagggcagaatcagcctgttttgaatatcacaaacaaacaggctttcattactgctcagaatcattgctatgccttggacaacaccctccctgctggctggaaaccactttttgtgaatgtcaacgatcaaacaaatgaggggattatgcatgagagcaaacccttcttcgctgtgcagttccacccagaggtcaccccggggccaatagacactgagtacctgtttgattcctttttctcactgataaagaaaggaaaagctaccaccattacatcagtcttaccgaagccagcactagttgcatctcgggttgaggtttccaaagtccttattctaggatcaggaggtctgtccattggtcaggctggagaatttgattactcaggatctcaagctgtaaaagccatgaaggaagaaaatgtcaaaactgttctgatgaacccaaacattgcatcagtccagaccaatgaggtgggcttaaagcaagcggatactgtctactttcttcccatcacccctcagtttgtcacagaggtcatcaaggcagaacagccagatgggttaattctgggcatgggtggccagacagctctgaactgtggagtagaactattcaagagaggtgtgctcaaggaatatggtgtgaaagtcctgggaacttcagttgagtccattatggctacggaagacaggcagctgttttcagataaactaaatgagatcaatgaaaagattgctccaagttttgcagtggaatcgattgaggatgcactgaaggcagcagacaccattggctacccagtgatgatccgttccgcctatgcactgggtgggttaggctcaggcatctgtcccaacagagagactttgatggacctcagcacaaaggcctttgctatgaccaaccaaattctggtggagaagtcagtgacaggttggaaagaaatagaatatgaagtggttcgagatgctgatgacaattgtgtcactgtctgtaacatggaaaatgttgatgccatgggtgttcacacaggtgactcagttgttgtggctcctgcccagacactctccaatgccgagtttcagatgttgagacgtacttcaatcaatgttgttcgccacttgggcattgtgggtgaatgcaacattcagtttgcccttcatcctacctcaatggaatactgcatcattgaagtgaatgccaagatgtccccgaactctgctctggcctccaaaacgactggctacccattggcattcattgctgcaaagattgccctaggaatcccacttccaggaattaagaacgtcgtatccgggaagacatcagcctgttttgaacctagcctggattacatggtcaccaagattccccgctgggatcttgaccgttttcatggaacatctagccgaattggtagctctatgaaaagtgtaggagaggtcatggctattggtcgtacctttgaggagagtttccagaaagctttacggatgtgccacccatctatagagggtttcactccccgtctcccaatgaacaaagaatggccatcgaatttagatcttagaaaagagttgtctgaaccaagcagcacgcgtatctatgccattgccaaggccattgatgacaacatgtcccttgatgagattgagaagctcacatacattgacaagtggtttttgtataagatgcgtgatattttaaacatggaaaagacactgaaaggcctcaacagtgagtccatgacagaagaaaccctgaaaagggcaaaggagattgggttctcagataagcagatttcaaaatgccttgggctcactgaggcccagacaagggagctgaggttaaagaaaaacatccacccttgggttaaacagattgatacactggctgcagaatacccatcagtaacaaactatctctatgttacctacaatggtcaggagcatgatgtcaattttgatgaccatggaatgatggtgctaggctgtggtccatatcacattggcagcagtgtggaatttgattggtgtgctgtctctagtatccgcacactgcgtcaacttggcaagaagacggtggtggtgaattgcaatcctgagactgtgagcacagactttgatgagtgtgacaaactgtactttgaagagttgtccttggagagaatcctagacatctaccatcaggaggcatgtggtggctgcatcatatcagttggaggccagattccaaacaacctggcagttcctctatacaagaatggtgtcaagatcatgggcacaagccccctgcagatcgacagggctgaggatcgctccatcttctcagctgtcttggatgagctgaaggtggctcaggcaccttggaaagctgttaatactttgaatgaagcactggaatttgcaaagtctgtggactacccctgcttgttgaggccttcctatgttttgagtgggtctgctatgaatgtggtattctctgaggatgagatgaaaaaattcctagaagaggcgactagagtttctcaggccacgccagtggtgctgacaaaatttgttgaaggggcccgagaagtagaaatggacgctgttggcaaagatggaagggttatctctcatgccatctctgaacatgttgaagatgcaggtgtccactcggagaatgccactctgatgctgcccacacaaaccatcagccaaggggccattgaaaaggtgaaggatgctacccggaagattgcaaaggcttttgccatctctggtccattcaacgtccaatttcttgtcaaaggaaatgatgtcttggtgaatgagtgtaacttgagagcttctcgatccttcccctctgtttccaagactcttggggttgacttcattgatgtggccaccaaggtgttgattggagagaatgttgatgagaaacatcttccaacattggaccatcccataattcctgttgactatgttgcaattaaggctcccatgttttcctggccccggttgagggatgctgaccccattctgagatgtgagatggcttccactggagaggtggcttgctttggtgaaggtattcatacagccttcctaaaggcaatgctttccacaggatttaagataccccagaaaggcatcctgataggcatccagcaatcattccggccaagattccttggtgtggctgaacaattacacaatgaaggtttcaagctgtttgccacggaagccacatcagactggctcaacgccaacaatgtccctgccaacccagtggcatggccgtctcaagaaggacagaatcccagcctctcttccatcagaaaattgattagagatggcagcattgacctagtgattaaccttcccaacaacaacactaaatttgtccatgataattatgtgattcggaggacagctgttgatagtggaatccctctcctcactaattttcaggtgaccaaactttttgctgaagctgtgcagaaatctcgcaaggtggactccaagagtcttttccactacaggcagtacagtgctggaaaagcagcatagagatgcagacaccccagccccattattaaatcaacctgagccacatgttatataaaggaactgattcacaactttctcagagatgaatattgataactaaacttcatttcagtttactttgttatgccttaatattctgtgtcttttgcaattaaattgtcagtcacttcttcaaaaccttacagtccttcctaaggttactcttcatgagattcatccatttactaatactgtatttttggtggactaggcttgcctatgtgcttatgtgtagctttttactttttatggtgtgattaatggtgatcaaggtaggaaaagttgtgttctattttcttgaactccttctatactttaagatactctatttttaaaacactatctgcaaactcaggacactttaacagggcagaatactctaaaaacttgataaaattaaatatagatttaatttatgaaccttccatcatgtgtttgtgtattgcttctttttggatcctcattctcacccatttggctaatccaggaatattgttatcccttcccattatattgaagttgagaaatgtgacagagcatttagagtatgaattc

SEQ ID NO: 3 - DNA, 2732 bp, human
aacaacatcctgggattgggacccactttctgggcactgctggccagtcccaaaatggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaattggacgggagacagagtgacgcactgactcacctagaggctgggacgtgggtagggatttagcatgctggaaataactggcagtaatcaaacgaagacactgtccccagctaccagctacgccaaacctcggcattttttgtgttattttctgactgctggattctgtagtaaggtgacatagctatgacatttgttaaaaataaactctgtacttaactttgatttgagtaaattttggtttt

SEQ ID NO: 4 - DNA, 288 bp, human
  misc_feature (17)..(17): “n” may be any nucleotide
cttatctaaaagagganctncaggtctcaaccntgccagtcacaccnaattaatgtccttcacaaaaataancagcatatgttccctttcaatttgagttcagtgagctcacagcaaaatttaccttttaattttnttcagcaaatccaagacgaatatacaaaggatgagattagataaagatttcagtttccngtatgccaccgntgccgccaattttccaaaaaagcctggctcctcttttcctgttcctccatccaagcccccaaagatctctaaccagaatta

SEQ ID NO: 5 - DNA, 2251 bp, human
aggatgtcttctggcaatttcatataagtattttttcaaaaatgtctcttctgtcaaccccacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgtagctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttaagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatctacatttaaaagcatctcagcctaccatgagaataagagaaagaaaatgaagatcaaaagcttattcatctgttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttctgtgggctaattaaataaatcactaatactcttctaagtt

SEQ ID NO: 6 - DNA, 14776 bp, human
cccccattgaaaaattgtctttctgatctttataaacaattatttaatatccagtaaaatcttctctatattgctttactagtgagttctattaaaattttgaagcacagaaaattcccctacagtataaagtatccccagtcacagagaagacaggggttttgcaatgatttctagaatagtgcaatttttatgcaagaacctaatataacacaaaaattatagcccgattttatttgtgggtatagatgcaaaattactaaaaatactattaacaagttgaatccttagggtgttaaaagagtatcactccatgaacgagttggttgtgatgtggaactatgaggtacttttatgatacaatataaaaatttatggtaattttatggtacattgtgagacagtgttttcttctagcatcatactagcaggtctatggagaaaaatcacaggattgtctcaatcaaaaaaagatttcattaacccaactctcatccctgataaacactgttagttatctagagaaagaagaaaattgtcccaatacagtcacctctttgccacacccagccaacagcagacgtgatggaagcctgaagaacaccctgccacgggcacaggcagaggcacaggcaccctgtcgtcctgattatttcaccttgtcacgggcagaggcacaggcaccctgtcgtcctgattatttcaccttgtcacaggcacaggcaccctgtcgtcctgattatttcaccttgtcacaggcacaggcactctgtcgtcctgattatttcaccttgtcacgggcagaggcacaggcactctgtcatcctgattatttcaccttgtcctagagtgtcctgccaatgggacagatgcaaaacaaataaaagccccggcttctgaaaagaagcacacagaaatgtcattattttcaaacgaggtgttcccgtatataaaatttgatgttggttgggcatctaacagtattatggccagaggactcagaccacagctgcatccctgtgaggcacagactctccagggcacgcgggtcccgctgggatgtgcacactcaggtgagctgcacagacaaggtgtcctcagcccaggggagccagaggcctgctctgcctctccaccctgatgcttcctgttctcaccccaccaaagccaaggcttcaatttcagtctgtggggagctgactctgctgctctcaagcactagaagaaggaaccagtaatcgaggaaacttgtggaccccaatggtgtctgtcccggccaggcctggctgggcccacacaggacaacagggttcaggggtctggacagctgtttctgcccagggaattgtccctgccacctcacactggccactggaaaggaaagagaggaggaggcggcaggctaacccacccgtgagccagtcgagtctacattgtcagttctcacctcgaggggtgccaaaaaccagagggaagcaaaggcccctgaagcctctgccagaggccaacgccccttcttggttcaggagaggtgcagtgttaggtgcagcacaaccaatgacttgcttatgtggctaataaattgtcaagagaaaaactgggttagaatgcaatatatagtatgtagtctcatttttgtataaatacaagtatagaatggcataactcaaaatccacaagtgatttggctggattgtaaatgacttttattttcttcatttctcatcatattttctattatacataaagattcattgttaatataaaagtacaaaattgcaacctatgaattaagaacttctatatattgccagttagaagacagaatgaaaaacattctcttcattctaaccacacacacaaaaaactccacaaaatacctatggactaccttcatagaaggtggaagagggtctgtatgaagaaaatgcttaatacatgaaagaagaagctagtcaatgtggaggtctattgtgcgccgggatcaacaaagacaagatatgtttaaaatggtgttctaaatttaccctaatgtaaaacaaatccaataaaactctaatgtgattttttaagaatttaaatttggaataattccaaagaacaatttttcttaatttctacagccagaatatatacctttaaaaaaaatgaaaacagagattaactttctcagaattggttgactcactctttccttttatttttcttccatggaattttccagttaacttgagaaagtggaatcgaattccgatgttgaattttccttctggccccattcatgtggcaggtggtgattcaggtactactgggggctgctcagacaaacctcctcatcagacatcaagaggctgttgcaccaggagggccggtaccgtgtctagaggtggtcggcatggggttggagttgtattacataaaccctactccaaacaaatgcatggggatgtggctggagttccccgttgtctaaccagtgccaaagggcaggtcggtacctcaccccacgttcttaactatgggttggcaacatgttcctggatgtgtttgctggcacagtgacaggtgctagcaaccagggtgttgacacagtccaactccatcctcaccaggtcactggctggaacccctgggggccaccattgcgggaatcagcctttgaaacgatggccaacagcagctaataataaaccagtaatttgggatagacgagtagcaagagggcattggttggtgggtcaccctccttctcagaacacattataaaaaccttcctttccacaggattgtcctcccgggctggcagcagggccccagcggcaccatgtctgccctcggagtcaccgtggccctgctggtgtgggcggccttcctcctgctggtgtccatgtggaggcaggtgcacagcagctggaatctgcccccaggccctttcccgcttcccatcatcgggaacctcttccagttggaattgaagaatattcccaagtccttcacccgggtaagagaaatagtgttgattttagggagaataactcagcaattggatctggtatgtgtgtattcaactcatttgcagacaaattgtggttgttcaataccagcctgttgtgaattacctgaattgatagcatcctggagcgacactcaaaatgtgtcgcctgtggtgcagctggagcccggagcctgcgtgccaggccccggaggcccccgccgtgccttgtcctggggctgatgatggggaggccggcgaggccgggctgctgcgacgccaggataaccgggctggcggccagatgcgcactcgctgggcgtccgcctgtgtttgccaaagcacgagttgaaacgtgaagtgttgggccagcccgtgtggcaccaatacctgccgcctacgactgttgtgaacactgaatgggccaacaaacctaaacgttaaatgaactgataacgccgtcagcacggagcaggcgctgggtgtttgcgctcttgcgcgtgcgctgctgtggggcgcaggctgacggcgggcgggggtcgcctgctccagctcgggctcccgcgccagaaccgggtccagaaccttgattccggaagcgggcaacggggtggttggtgggcgcgcctgagggaagggacgtgaggagccggagtccgcggagttgccgcggagttgtccgcggagtccaggcgggtggggagcagagcagctggaaccccccgagcgccctgcagacgcagcagcctcttgaggggagggtctcccccacctcgggctggacaaagacagcttttccccacgtccctctgggttctctagagcaacagcaatacccgcccggcaggtgtggcttagagccccgcacctcctcgccgcgcgcgggcctgacttctagccacgggtctccgcagttggcccagcgcttcgggccggtgttcacgctgtacgtgggctcgcagcgcatggtggtgatgcacggctacaaggcggtgaaggaagcgctgctggactacaaggacgagttctcgggcagaggcgacctccccgcgttccatgcgcacagggacaggggtgagtccgcgtccctggcacggagcggggggtgcataacacgccccgggacagttacgggcgctagccacgtcggcgatggccaaataataaactaacagtaatattatagtaatagcatccgaaggatgagatcaggattaggcgatggcccccgcgcgttgcctgccgagcgaggcgcactgagtcgcccaggaatccggcctctcggcgactgtgcgggagagttttatggggatgggcggggctgcttctgagcaggagtcgccgcccccacccccaccgttccgcctctgggccgcaggctcctcccgggagcgctttcccctcctgttcaaccgccggggtacaggtggcttcgtccaccgaggtcccctcacccacgctgaggcgtcggaagctgcggacactgctcgcttcagggctttgctcagctgcagctggtgacctccagagagggagtctctgatgtcccgctggggtggatgtcctgagaccgggaagggggaagagacccactgaaatcctatctcccagcctcacctctgctgtctcctccacgcttcctgtctccagagccccgagttcagcataagcagaaagcggcctgttccctctctagggagaggagggttgcggtctggaggtctggctcgtctttatctgcgcattctcccagcctcctggcttcagacctcagcgaggcggcggctgcggccggctctcctcttcctgcctgcagacctggcctgctgcttctttctccttcctccctccctgcctgccctgcggtttcaaagtagattagaaataacagtgtcccacatggaagcctctacttcttcctgggtcaactttgatgacgaggctccagaaaacctttgcaatgctgtgtggaatttttaaatcggtgagctcgtgctcttgccctatttatttgtccagcgtacatttctgaacattgtgaacgtcgaatgggccaacaaatctaaaaattaaatgagctgataaagaacgccgtcagcacagagcagacgctgggtgttcgcgctcttgagcgtgcgctctgcggggcgcgggctggtggcgggcgggggtcgccggctccagctcaggttcccgcgccaggaccgcgtccagaaccttgtctccggaagcgggcaacggggtggttgtatcacaattagtggcatttggttttccttcttctgcattgtgggttttacttctctggggttgccaaaaacaaaattaaccatctcagtccttgtcgttaacgcaggagaagcattactggaggaggctctggggttctgtggttgaggagctcagttctggttccggggagcccttatctgccacccacgggtccaaggcacagtcggaggcagcagggaggggagcggaattcacatcaacacagatggggctcaaggggactttgctgcctctgcctggagggtctaaagtttcattttcatatgacccgcagggcgcagactggcggaaaattagcagagccctgggcatgggctgcacctggccttaagggacaatgatggaaatattccttattagcacaatactgagcacaggctgtgtgataatgtgtcaagggaactgcagacatcctttcagaaaaagttcataaaacggagaaagtttggttcccaacctagatttttaacctgttgaactctgtctaaatgggtcatctcgggatgtcctccactcaacatgaccacagtctgcccctctgtcccacctgtctcctcagtccttcctccccacctttcaggatgaaatgaaaccctcagtccagctgcacccctgccccacccacctcatctcatgtgccctcccgcccctctcaggccggacagccttgcttctggaacacacgagcacagcttcaccaggcactttctgagcaccctgcaggcgcctcccaggagtggtcagtggtcaatcagctaatgaagctgcataggacatgacccttgtttaccgcagaatgcccagagctggcaggatgtcttatatgcaggaagtacccaaaatgtatttattgaggaagtgatgatggataagaggaagacggagagcgagggagagaggggctaggggccctgcggtgtaaagggggtgtggctgggagtgtgcaggggaacagggatcatttcaaggttcctatctgggagaaaataaaaaggtttacagttagttgagataagcgtgggaatatgcgaacatttttaaagaataaaaagtttagctttaaatttgttgattccaaatgtgttcatactctcgggaggatccatcaagcaactcttgggaggagagacagggcagggcaggccttgacagctcagaagggcgcagtagggacagttcttggttttcccagctctgatgctttgcacagtcgcttgtgtgacctgcaagattttagtgaagaaacttgctgtggagtcggaaagctgcaagttgaggtgtgtgtggtgtgagggttaaaaatctgtgagaacagaatgaatggcttttcaagaatgttgtcgatagataggaaagaggtgggaggtgttcttggagtggccatatgtggttttatgtagcatggggaagactcagcagaaaggaaaaagaaagaaggtaaattgacagcatgaagtagagcacccaggagaggctacatgtgatgaagaaaccacagtgcagactgtgaggaccccagaaaggctcctccccaaaacctgaccagtggccggtgctggcagctcccaggctgggacaccctctgtctctctgtccctctgccccctctgtcacttctttatacacctgtaaatcctgccctgctctccaaggccctctgtagcccatttctccccaaaatgggtatttagaataaccttctgctggcccctctgccttaggaatcatttttaataatggacctacctggaaggacatccggcggttttccctgaccaccctccggaactatgggatggggaaacagggcaatgagagccggatccagagggaggcccacttcctgctggaagcactcaggaagacccaaggtgcgtatctgctgcctagcagggcccagtcctcttgcagaccagcggtgtggggagccctggctgggactcctagactgcatctgaaccacagggacctacggacaaggagagggtctcgtgagtccccagatactgcattttacaactctaggttccagctacacagttcagggagcaagggtggccattaaacacgtgacttgtatcctaaatactgttgaaaagcaaaggaaactcaaacaggttcagacattcactatctttcgtaaactggcagttttcagggcaccttctcacaggccttggtgaacctcagtgggtgactgagcaggtggaggagtctcctcacccccatcttctggttgccctgactgcctgttttgtaggccagcctttcgaccccaccttcctcatcggctgcgcgccctgcaacgtcatagccgacatcctcttccgcaagcattttgactacaatgatgagaagtttctaaggctgatgtatttgtttaatgagaacttccacctactcagcactccctggctccaggtgaagccactttcctctttcatcagtcatcaactgtagagtttacgttagaaaaagaaggaaaatttgggttatatgtgatagacaggactgcaaaagccaaacaacatagcttcgaggggtgtttgattagacagcccaaatattcctcccagagacatctctggggccccacgcaccccctttcctaacgtcaggatgtgtatcgacctgtgtgtgcacatttgccatgcagagtttgcactgctgaggagaatggtgcccaagaaggacactgttgacccaaaatattccaaataaacaatgattacagccacaaattcaggtttggagaaagttgttggtccaacacacacaattatgttgcatccagaaaaaagtagtaaaatatttttttccctctctagctttacaataattttcccagctttctacactacttgcctggaagccacagaaaagtcataaaaaatgtggctgaagtaaaagagtatgtgtctgaaagggtgaaggagcaccatcaatctctggaccccaactgtccccgggacctcaccgactgcctgctcgtggaaatggagaaggtaggctcggcctcccatgatgtgggctctccggggtgggcagagaatgcacaatttcagatttacagagtgagctgcacttgctggtgtccagacctcccaccgcagcatgctctgagtttcatacacacactcttggcttcagcatgaccactggacgcaagtcagcctgcctggctgccaagctggcctggggtttggggcacatgggcgggacgcttagctctctccaggccctgctgctcaaccctttctagtctgcagactttgagaattgcattttgtctgaggagaagccctcagccttccttgtgggcatgcactccccaactgtgcgcacgtgcaggacttccaggcctccccagcttcatccacctgcaggtgctcaggatcctgatcccctgcccccttcccaccttggtgaaacttcttgtatccttgtcttgtcctttcctatggcttgtggctcaagaacaaatgtggagcccacactgatttcccaggactgtctgagcatcttctccaccagtttggcccctcgtggcagcagacactagccctgtagcaggaggggttagcaggagccgtttagctcctgcctgagctatgaccaaggtcagggggatctcacctctcccaggatggccctcatgctgtggagggagacagagccctggcctgccctcagcagatttctgggagcctcagtttccctggctgtgagtggagatgactctgtctgtcacagctccaagtcacagttccactgggagagcctcttggacactgtctcctgtgtccctgtggagctgggaggtggctggttctgtgctgaaaggagacaagcagccccttctctccggtctgtctccggtatcacaggaaaagcacagtgcagagcgcttgtacacaatggacggtatcaccgtgactgtggccgacctgttctttgcggggacagagaccaccagcacaactctgagatatgggctcctgattctcatgaaataccctgagatcgaaggtaggcaagtgactgaagggacaccgtgcgtgcggctgcatctccctggatggccagccttgcacattttaggctgcagctttctgtctgaagctgcttgttaaccctcatggtgatgtggtgagatggctggatgcactgctgtgaggggaggtgttatggtctgtgctgaacactggtactcttgcacactggttggtccataccccactaagacacccctggttgcagaaaagaacatcccaacaccagagtggagagaggtggcagggtctgcattctgctccataaataacctctttatgacagagaagataatgtcccagttccccccaagtaagacctggtcttctaggcagagcaggtggggaggttggagctggaggggagggtccttgctggggcgtcttcctcaaatgcggacgtgaggagggaagtccaggaagaagcagctacagctccccctggacccttgtcgttccttccacagggctcctcccagcggcacctggggcagctgggactctgtgcctggaggaggtgtgaaaggtctgggtctaggtgggcagagggtcatgccctgagaaacacccatctgggccaagtagaggtgatgtgagggcaccgcatgcaaacaggccagtcagggttgggtccaagtaaaggggaggaaagggagctgcagcctggctggagagtgccggggggcccagagcccctgcctctcgctgggctggaaacagggctgggcagcctctgcccgaggcagttcacagcctgagtggtgtgtgccgccctcctcctgaagctgctgctaatggtcacttgtggtcttaaggctcgtcagttcctgaaagcaggtattataggctatgaagttatttcccccaagaaagtcgacatgtgatggatccagggtcagaccctggcttttcttgttctttccttcttcttcttctttttatttatttattttttttttgaggggacagggtctcactctgttgcccaggctggagtgcggtgatgcaatcatggctcattgtagcttctacctattgggctcaagcgatcctcccacctcagcctcccaagtaactgggccacaggtgcacaccaccacacccagctgattaaaaatttaaaaaaattattttggctgggcacagtggctcatacctgtaatcctggcactttgggaggctgaggcaggcgatcacgaggtcaggagttcgagaccttcctggccaacatgatgaaaccctgtctctcctaaaatacaaaaaagtagccgggtgtggtggcacgcgcctatagtcacagctactcaggaggctgaggcaggagaatcgcttcaacctcagaggcacagggtgcagtgatccgagattgcaccccactgcactctagcctgacaacagagcaagaatcagtctaaaaaaaaaattgtagagacaagttgttactatgttttgtaggctggtcttgaactcctgggctcaagtcatcctcctgccttggcctcccaaagtgctggggttacaggtgtggccaccgtgccccatccctggcctttgctttttcaatcacatggaaatgtgaagggtgaaggagccaaaagtttagggaaggaatcattgtatggatctgcagtgattataagagaactttcgactactctgcactaggggaaccatggaatcaaaaaatgttttaaattattatttatgaggaggttccaatatagacaaaaggaaaataaatatgattgacatgtatatatccattgccaaattgaacgtttattaacattttgcgatacttccatcagagctcttaaaaagaaaatgtgttacagagccagccaaagtctacctcctcacatctccccacctctctcaccagaaatggcttcagaattgctgtgtggctttgcacttttaacagttgttaattatcagcacagtattcatattattgctgtatgtgtttaatattttacctgggtactgtacataacattttgcagcttggttttttcactcaacatatgatgatgttccatgggaactccaaacacggggaggctaggcgacttgctcaaggcagctgttacctctgtcagaaagacagaggctttcagattcaagaagtagaccctgcatgtctgattctgttctgtaaacccccttcatactcagaagcatgcaataaacaagcctggggtaattatcaatgcaaaggttaccctcccagaagaaatttccaaaacactttcattattctctgctcttgacatgaagagaactgaataagccatcatcaactgagataatggatgccaaaacatccagtaaataacctcatagagcttagctctcactaagtttttggagcattttccagtaattcaaaggacctggggaaccttaagcactgcttaggatgctccataaacatcttctgcgtgggtaggggagtggatggatggctggatgggtgggtggatggacggacggatggatggatggatggatggatggatggttggatggatgggtgggtggatggatggatgggtcaatggatgtgtggatggatggaagggtgggtggatgggtggatggctggctggttgggtgggtgggtggatggatgcatgggtggatggatggaggatggatggatggatggaggggtgtatagatggaggggtggatggatgtgtaggtgggcagatggataaaagcgtgattgaatagatgggtggatgatgggtggatgcccaactggccaggaaccaatccctgaaatttgtcccattcatatcttggcagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaaggttaagcaatgagcctgcagcacacagcatgaacaccatcctatcactaatcgccttcctgccagggagcaggatgggggccccaagacccttccctttggcaggggtcactgaggggaagggctggccccactcccaccctgtgggatactgcatctccaggagtgctcacattggcctggtgaccagagaggtggaggaaatctggaaaagagcctcagcagatagtgcctgggactgtagtgaattctaatgccaggaacaaactatcacaaccagccctggggttaatcctgtgagaagattagggctttcatcttcatttagacctgacccctgactgctttctatctaatccttcactaagcaactccttcaactcgaaatatactatcctatatagcataatattcaaaacaacattcttcactgggggtttccagatgaaagcccacattttgttaacatgactcactgagacagtctttgtttctcctagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggtgagaaagatcagaggcagtaccttcccttgaggagcagcccacactcctcatctcccctccacatgtgctctgccctcgtcccaggcacccactgacaccccaaacctcactgtgtgccctgtttctattgacaacatgacccaaatgtgctcttccctgttcagagaagttacataacatcttttagcagcaatcctgggaatgaagtgttgtaggtggattttttttttcccaaagactagacattttacatcattcattgctaaattttgtttctattttaacaagacttagtgaaaagctctcaaagccatattacccaattctccctaattttaaaccagagctactaaacaaaacctaacctttggttacctagaatcatcacaggaagcatcaaagccttcctgggatgtgactcagtgattttctttgaggcacttgtcctccttcccagggcctcatcttagggattgttgtgggaagatcatacaaccaactccatacttttcacacccagtgctggagccccagcttctaacagggcactatttccctcctgtaggcatcactgatgagcactgggggtgccttctttactgggcagacatggtcttcccaacttaacaccggtttttgcagttgagctctggataattgagattgtatgaaggctggtccccgaattagtcagtgtcgctggtatccttccactcaagtacattttgtgcttcttttaataggcagagaggggtgagtcctgccctgtgatggccgtttgcccacagcctcctcctccccgcttcccctagtctcactgttaacagtgtcgtgtctctgaaactccctcagtgtctcatcaataccattgttacttctaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaaccccccgctttcaaacaagatttcgaattgtttgaggtcaggatttctcaaactgattcctttctttgcatatgagtatttgaaaataaatattttcccagaatataaataaatcatcacatgattattttaactatatgttaagtcatggaatatcttaattgtttaagtgattctcacagagaggtttttttttttttttttttttttttgagagttttgctcttgttgaccaggatggagtgcagtggcatgatcttggctcactgcaacctctgtgtcctgggttcaagtgattctcctccctcagcctcccgaatagctgggattacaggcacccaccaccatgccagctaattctttgtatttttagcagagacagggtttcaccatgttggtcaggctggtcttgaacccctgacctcaggtgatccacctacctcggcctcccaaagtgctgggattacagcatgagccaccgcgcccagccagagagaggttttaaatatatatgtttactttaatattaagttataacataattttcatgttattgaaaagctcttccatctaggatcacaccacttcagtgtcagaatcatattgaggtggggaatttgtattagtcaggtttctctaaagggacagaaacaataggatagatgtatatacgaaagggagtttattaggagaattgactcacatga

SEQ ID NO: 7 - DNA, 882 bp, human
cggccaggcttgcgcgtggttcccctcccggtgggcggattcctgggcaagatgaagtgggtgtgggcgctcttgctgttggcggcgtgggcagcggccgagcgcgactgccgagtgagcagcttccgagtcaaggagaacttcgacaaggctcgcttctctgggacctggtacgccatggccaagaaggaccccgagggcctctttctgcaggacaacatcgtcgcggagttctcggtggacgagaccggccagatgagcgccacagccaagggccgagtccgtcttttgaataactgggacgtgtgcgcagacatggtgggcaccttcacagacaccgaggaccctgccaagttcaagatgaagtactggggcgtagcctcctttctgcagaaaggaaatgatgaccactggatcgtcgacacagactacgacacgtatgccgtacagtactcctgccgcctcctgaacctcgatggcacctgtgctgacagctactccttcgtgttttcccgggaccccaacggcctgcccccagaagcgcagaagattgtaaggcagcggcaggaggagctgtgcctggccaggcagtacaggctgatcgtccacaacggttactgcgatggcagatcagaaagaaaccttttgtagcaatatcaagaatctagtttcatctgagaacttctgattagctctcagtcttcagctctatttatcttaggagtttaatttgcccttctctccccatcttccctcagttcccataaaaccttcattacacataaagatacacgtgggggtcagtgaatctgcttgcctttcctgaaagtttctggggcttaagattccagactctgattcattaaactatagtcacccgtg

SEQ ID NO: 8 - DNA, 2452 bp, human
gtggacttgttgcagttgctgtaggattctaaatccaggtgattgtttcaaactgagcatcaacaacaaaaacatttgtatgatatctatatttcaatcatggaccaaaatcaacatttgaataaaacagcagaggcacaaccttcagagaataagaaaacaagatactgcaatggattgaagatgttcttggcagctctgtcactcagctttattgctaagacactaggtgcaattattatgaaaagttccatcattcatatagaacggagatttgagatatcctcttctcttgttggttttattgacggaagctttgaaattggaaatttgcttgtgattgtatttgtgagttactttggatccaaactacatagaccaaagttaattggaatcggttgtttcattatgggaattggaggtgttttgactgctttgccacatttcttcatgggatattacaggtattctaaagaaactaatatcaattcatcagaaaattcaacatcgaccttatccacttgtttaattaatcaaattttatcactcaatagagcatcacctgagatagtgggaaaaggttgtttaaaggaatctgggtcatacatgtggatatatgtgttcatgggtaatatgcttcgtggaataggggagactcccatagtaccactggggctttcttacattgatgatttcgctaaagaaggacattcttctttgtatttaggtatattgaatgcaatagcaatgattggtccaatcattggctttaccctgggatctctgttttctaaaatgtacgtggatattggatatgtagatctaagcactatcaggataactcctactgattctcgatgggttggagcttggtggcttaatttccttgtgtctggactattctccattatttcttccataccattctttttcttgccccaaactccaaataaaccacaaaaagaaagaaaagcttcactgtctttgcatgtgctggaaacaaatgatgaaaaggatcaaacagctaatttgaccaatcaaggaaaaaatattaccaaaaatgtgactggttttttccagtcttttaaaagcatccttactaatcccctgtatgttatgtttgtgcttttgacgttgttacaagtaagcagctatattggtgcttttacttatgtcttcaaatacgtagagcaacagtatggtcagccttcatctaaggctaacatcttattgggagtcataaccatacctatttttgcaagtggaatgtttttaggaggatatatcattaaaaaattcaaactgaacaccgttggaattgccaaattctcatgttttactgctgtgatgtcattgtccttttacctattatattttttcatactctgtgaaaacaaatcagttgccggactaaccatgacctatgatggaaataatccagtgacatctcatagagatgtaccactttcttattgcaactcagactgcaattgtgatgaaagtcaatgggaaccagtctgtggaaacaatggaataacttacatctcaccctgtctagcaggttgcaaatcttcaagtggcaataaaaagcctatagtgttttacaactgcagttgtttggaagtaactggtctccagaacagaaattactcagcccatttgggtgaatgcccaagagatgatgcttgtacaaggaaattttacttttttgttgcaatacaagtcttgaatttatttttctctgcacttggaggcacctcacatgtcatgctgattgttaaaattgttcaacctgaattgaaatcacttgcactgggtttccactcaatggttatacgagcactaggaggaattctagctccaatatattttggggctctgattgatacaacgtgtataaagtggtccaccaacaactgtggcacacgtgggtcatgtaggacatataattccacatcattttcaagggtctacttgggcttgtcttcaatgttaagagtctcatcacttgttttatatattatattaatttatgccatgaagaaaaaatatcaagagaaagatatcaatgcatcagaaaatggaagtgtcatggatgaagcaaacttagaatccttaaataaaaataaacattttgtcccttctgctggggcagatagtgaaacacattgttaaggggagaaaaaaagccacttctgcttctgtgtttccaaacagcattgcattgattcagtaagatgttatttttgaggagttcctggtcctttcactaagaatttccacatcttttatggtggaagtataaataagcctatgaacttataataaaacaaactgtaggtagaaaaaatgagagtactcattgtacattatagctacatatttgtggttaaggttagactatatgatccatacaaattaaagtgagagacatggttactgtgtaataaaa

(19) United States
(12) Patent Application Publication
(10) Pub. No.: US 2004/0248142 A1
(43) Pub. Date: Dec. 9, 2004

(54) Method for detecting hepatocellular carcinoma

(75) Inventors: Moritoshi Kinoshita, Itano-gun (JP); Masahiko Miyata, Kure-shi (JP)

Correspondence Address: SUGHRUE MION, PLLC, 2100 PENNSYLVANIA AVENUE, N.W., SUITE 800, WASHINGTON, DC, 20037, US

(73) Assignees: OTSUKA PHARMACEUTICAL CO., LTD.; NATIONAL HOSPITAL KURE MEDICAL CENTER

(21) Appl. No.: 10/625,899

(22) Filed: Jul. 24, 2003

(30) Foreign Application Priority Data
Sep. 13, 2002 (JP) 2002-268369

(51) Int. Cl. C12Q 1/68

(52) U.S. Cl. 435/6

(57) ABSTRACT

The present invention provides an effective method for detecting hepatocellular carcinoma in a tested tissue, which comprises the step of measuring the expression level(s), in the tested tissue, of at least one gene selected from the group consisting of plasminogen gene, EST51549, retinol-binding protein 4 gene and organic anion transporter C gene, and a method comprising the step of measuring, in the tested tissue, the expression level(s) of at least one gene selected from the group consisting of plasminogen gene, EST51549, retinol-binding protein 4 gene and organic anion transporter C gene, and, in the tested tissue, at least one gene selected from the group consisting of aldolase B gene, carbamyl phosphate synthase 1 gene, albumin gene and cytochrome P450 subfamily 2E1 gene.

### BACKGROUND OF THE INVENTION

(1) Field of the Invention

The present invention relates to a method for detecting hepatocellular carcinoma in which expression levels of genes in a tested tissue collected from chronic hepatitis and other patients are measured.

(2) Description of the Related Art

It is believed that there are 500 million patients with viral hepatitis in the world. In South Asia in particular, 24.8% of individuals are infected with hepatitis B virus or hepatitis C virus, and 5% of these individuals suffer from chronic hepatitis. It is known that chronic hepatitis develops to hepatocellular carcinoma over a period of about 20 years.

Currently, abdominal echography, abdominal MRI, abdominal CT, angiography, biochemical test of tumor marker in blood serum, liver biopsy, etc., are known as methods of detecting and diagnosing hepatocellular carcinoma. However, there is no method that can effectively detect or determine hepatocellular carcinoma by measuring of the expressions of genes.

Carcinogenesis is one of the phenomena observed when a normal cell is affected by various outside factors and a change or alteration occurs in its genetic level, function of protein is affected by the change or alteration, and the normal cell functions are consequently destroyed. Many works have been reported of changes or alterations at the genetic level occurring in hepatocellular carcinoma tissues.

For example, it has been reported that the gene amplification of c-myc was in 33.3 to 36.4% of cases in hepatocellular carcinoma tissues (Oncology, 1999, 57, p. 157-163; Journal of Formos Medical Association, 1993, 92, p. 866-870).

It has been reported that point-mutation occurred in K-ras at a frequency of 0-16.7% (Anticancer Research, 1995, 15, p. 859-861; Oncogene, 1991, 6, p. 857-862).

It has also been reported that point-mutation occurred in p53 at a frequency of 23.1-50% (Cancer, 1994, 74, p. 30-37; Gastroenterlogy, 1999, 117, p. 154-160; Journal of Hepatology, 1993, 19, p. 312-315; British Journal of Cancer, 1999, 80, p. 59-66; Journal of Gastroenterological Hepatology, 1995, 10, p. 179-185).

Furthermore, in Rb and p53, loss of heterozygosity was observed at frequencies of 42.9-43.1% and 50-52.9%, respectively (Journal of Hepatology, 1993, 19, p. 312-315; British Journal of Cancer, 1999, 80, p. 59-66; Cancer Research, 1994, 54, p.4177-4182; European Jouranal of Cancer, 1999, 35, p. 1730-1734).

Underexpression of aldolase B has also been reported (Journal of Clinical laboratory analysis, 1994, 8, p. 144-148) and of albumin (Journal of Histochemistry and Cytochemistry, 1997, 45, p. 79-87).

It has also been reported that, in a rat liver carcinogenesis model, the expression level of the carbamyl phosphate synthase 1 gene decreases in proportion to the degree of malignancy of cancer (Scientia Sinica Series B, 1988, 31, p. 197-203).

However, these reports regarding the change or alteration of expression levels of genes are not an adequate basis to determine that precancerous conditions develop to hepatocellular carcinoma, and therefore an appropriate method for detecting hepatocellular carcinoma has not yet been established.

### BRIEF SUMMERY OF THE INVENTION

A primary object of the present invention is to provide an effective method for detecting hepatocellular carcinoma. The invention also aims at providing an effective means for detecting hepatocellular carcinoma.

The present inventors performed a thorough comparison of expression levels of genes in the livers of chronic hepatitis patients between cancerous regions and noncancerous regions. As a result, they found that there are some genes whose expression levels significantly decrease in the cancerous region. They conducted further extensive research and completed the present invention. The invention relates to a method for detecting hepatocellular carcinoma, a method for early detection of hepatocellular carcinoma, and a DNA chip for detecting hepatocellular carcinoma as described below.

Item 1. A method for detecting hepatocellular carcinoma comprising the steps of:

(a) measuring, in a tested tissue, the expression level(s) of at least one gene selected from the group consisting of plasminogen gene, EST51549, retinol-binding protein 4 gene and organic anion transporter C gene; and

(b) comparing the expression levels of the genes measured in (a) with the expression levels of the genes in a control that correspond to the genes measured in step (a).

Item 2. A method for detecting hepatocellular carcinoma comprising the steps of:

(a) measuring, in a tested tissue, the expression level(s) of at least one gene selected from the group consisting of plasminogen gene, EST51549, retinol-binding protein 4 gene and organic anion transporter C gene, and at least one gene selected from the group consisting of aldolase B gene, carbamyl phosphate synthase 1 gene, albumin gene and cytochrome P450 subfamily 2E1 gene; and

(b) comparing the expression level(s) of gene(s) measured in (a) with the expression levels of genes in a control that correspond to the genes measured in (a).

Item 3. A method for detecting hepatocellular carcinoma according to any one of Items 1 or 2, wherein the step (a) of measuring the expression level(s) of the gene(s) is performed by determining the amount of transcripts of the genes being measured.

Item 4. A method for detecting hepatocellular carcinoma according to any one of Items 1 or 2, wherein the step (a) of measuring the expression level(s) of the gene(s) is performed by amplifying whole or a part of the DNA to be measured and using cDNA prepared from gene transcripts as a template.

Item 5. A method for detecting hepatocellular carcinoma according to any one of Items 1 to 3, wherein the step (a) of measuring the expression level(s) of the gene(s) is performed by invader assay.

Item 6. A method for detecting hepatocellular carcinoma according to any one of Items 1 to 2, wherein the step (a) of measuring the expression level(s) of the gene(s) is performed by hybridizing labeled cDNA prepared from transcripts including the gene(s) to be measured with whole or a part of the immobilized DNA of the gene(s) to be measured.

Item 7. A method for detecting hepatocellular carcinoma according to any one of Items 1 to 6, wherein the tested tissue in the step (a) is liver tissue of a chronic hepatitis patient.

Item 8. A method for detecting hepatocellular carcinoma at an early stage that comprises the step of periodically measuring the expression level(s), in a tested tissue, of at least one gene selected from the group consisting of plasminogen gene, EST51549, retinol-binding protein 4 gene and organic anion transporter C gene.

Item 9. A method for detecting hepatocellular carcinoma at an early stage that comprises the step of periodically measuring the expression level(s), in a tested tissue, of at least one gene selected from the group consisting of plasminogen gene, EST51549, retinol-binding protein 4 gene and organic anion transporter C gene, and at least one gene selected from the group consisting of aldolase B gene, carbamyl phosphate synthase 1 gene, albumin gene and cytochrome P450 subfamily 2E1 gene.

Item 10. A DNA chip for detecting hepatocellular carcinoma in which whole or a part of DNA comprising transcribed region(s) of at least one gene selected from the group consisting of plasminogen gene, EST51549, retinol-binding protein 4 gene and organic anion transporter C gene is immobilized.

Item 11. A DNA chip for detecting hepatocellular carcinoma in which whole or a part of DNA, in a tested tissue, comprising transcribed region(s) of at least one gene selected from the group consisting of plasminogen gene, EST51549, retinol-binding protein 4 gene and organic anion transporter C gene, and, at least one gene selected from the group consisting of aldolase B gene, carbamyl phosphate synthase 1 gene, albumin gene and cytochrome P450 subfamily 2E1 gene.

### BRIEF DESCRIPTION OF SEVERAL VIEWS OF THE DRAWING

FIG. 1 compares the expression levels of genes in hepatocellular carcinoma tissues and noncancerous tissues after conducting electrophoresis.

GTVA and GTVC indicate anchor primers, AP indicates an arbitrary primer. A to D represent tested patients, in which A and B are patients infected with the hepatitis B virus, and C and D are patients infected with the hepatitis C virus.

Lane N and lane T are electrophoresis patterns of samples prepared from noncancerous tissue and cancerous tissue respectively. M is a molecular marker.

Arrows point the genetic bands after conducting electrophoresis in which expression levels of genes are lower in hepatocellular carcinoma tissue than in the control.

### DETAILED DESCRIPTION OF THE INVENTION

Hereunder, the present invention is explained in detail.

Representation of amino acids, peptides, base sequences, nucleotides, etc., by abbreviations in this specification is in conformity with the rules recommended by IUPAC-IUB, “Guideline for Preparation of a Specification or Equivalent Referring to a Base Sequence and/or an Amino Acid Sequence”(edited by the Japan Patent Office) and the conventions relating to the use of codes or symbols in the art.

(1) Genes Whose Degree of Expression is Lowered By Canceration

The present inventors performed a thorough comparison of expression levels of genes in the livers of chronic hepatitis patients between cancerous regions and noncancerous regions.

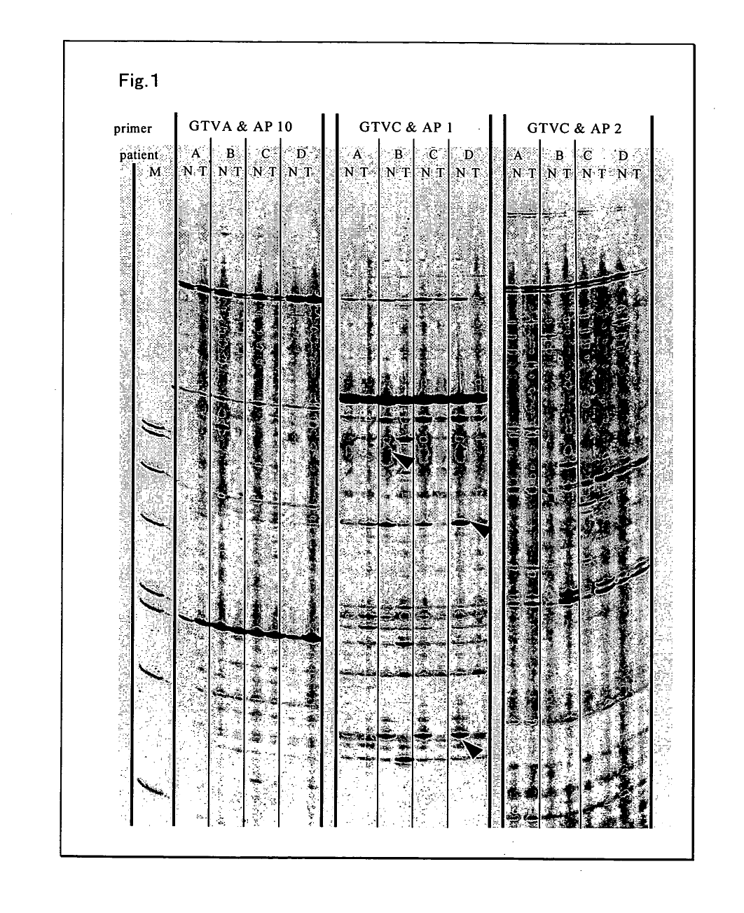

Specifically, a fluorescent-labeled cDNA library was synthesized from mRNA that had been prepared using a cancerous region and a noncancerous region of the liver of a chronic hepatitis patient, and then the library was subjected to separation by electrophoresis. The variance in the intensity of fluorescence between two tissues were examined, and genes significantly underexpressed in the cancerous region were selected as potential genes useful for detecting hepatocellular carcinoma (FIG. 1). The potential genes were then cloned to determined their base sequences. Furthermore, using a mRNA solution prepared from a cancerous region and a noncancerous region, the potential genes were quantified by a real-time RT-PCR method to confirm that they were actually underexpressed in the hepatocellular carcinoma.

Eight genes that were significantly underexpressed in hepatocellular carcinoma were selected.

The 8 genes were analyzed using the GenBank gene database, and it became clear that the 8 genes had the base sequences of Seq. Nos. 1 to 8.

The gene having the base sequence of Seq. No. 1 is a gene that codes aldolase B.

The gene having the base sequence of Seq. No. 2 is a gene that codes carbamyl phosphate synthase 1.

The gene having the base sequence of Seq. No. 3 is a gene that codes plasminogen.

The gene having the base sequence of Seq. No. 4 is EST51549 (GenBank Acc. No. AA345522) whose function is unknown.

The gene having the base sequence of Seq. No. 5 is a gene that codes albumin.

The gene having the base sequence of Seq. No. 6 is a gene that codes cytochrome P450 subfamily 2E1.

The gene having the base sequence of Seq. No. 7 is a gene that codes retinol-binding protein 4.

The gene having the base sequence of Seq. No. 8 is a gene that codes organic anion transporter C.

As shown in Table 1, the 8 genes were remarkably underexpressed in hepatocellular carcinoma. In particular, underexpression of plasminogen gene, EST51549, retinol-binding protein 4 gene and organic anion transporter C gene in hepatocellular carcinoma is a new finding of the present invention.

(2) Detection Method

The detection method of the present invention is characterized in that it comprises the step of measuring the expression levels of the genes that exhibit underexpression in hepatocellular carcinoma in a tested tissue.

As described above, the 8 genes exhibit remarkably lowered expression levels in cancerous tissue developed from hepatocellular carcinoma. Therefore, by measuring the expression levels of these genes and comparing with a control, it becomes possible to detect hepatocellular carcinoma.

In the present invention, measurement of expression level(s) of, in particular, at least one member selected from the group of 4 genes consisting of plasminogen gene, EST51549, retinol-binding protein 4 gene and organic anion transporter C gene is conducted. The measurement may be conducted by measuring the expression level of one gene out of the 4 genes, more than one of the 4 genes, or all of the 4 genes.

In the present invention, it is preferable that, in addition to the above-mentioned 4 genes, the expression level(s) of at least one gene selected from the group consisting of the four further genes, i.e., aldolase B gene, carbamyl phosphate synthase 1 gene, albumin gene and cytochrome P450 subfamily 2E1 gene be measured.

In addition to measuring the expression level(s) of at least one gene selected from the four genes consisting of plasminogen gene, EST51549, retinol-binding protein 4 gene and organic anion transporter C gene, by further measuring the expression level(s) of at least one gene selected from the group consisting of aldolase B gene, carbamyl phosphate synthase 1 gene, albumin gene and cytochrome P450 subfamily 2E1 gene, it becomes possible to conduct measurement or detection in a more accurate manner.

In particular, it is preferable to measure the expression levels of all 8 genes, i.e., plasminogen gene, EST51549, retinol-binding protein 4 gene, organic anion transporter C gene, aldolase B gene, carbamyl phosphate synthase 1 gene, albumin gene and cytochrome P450 subfamily 2E1 gene.

In the detection method of the present invention, as long as the measurement of the expression level(s) of at least one member selected from the group of 4 genes consisting of plasminogen gene, EST51549, retinol-binding protein 4 gene and organic anion transporter C gene is included, measurement of the expression levels of publicly known genes other than the above-mentioned 8 genes can be included.

The method for measuring the expression levels of genes is not particularly limited and conventional methods can be suitably used. For example, it is possible to employ a method in which the amount of transcript is determined or a method in which the amount of translated products is determined.

The method for determining the amount of transcript from a gene is such that mRNA is extracted from the tested tissue to determine the amount of the RNA products derived from the gene.

Extraction of RNA from the tested tissue and purification can be conducted by following conventional methods. Specifically, it can be conducted as follows: To the tested tissue, a solution containing phenol and guanidine thiocyanate is added. After dissolving or homogenizing, chloroform is added thereto, and the solution is then separated by centrifugation into an aqueous solution layer, which is the upper layer, and an organic layer, which is the lower layer. The RNA is dissolved in the aqueous layer, and therefore RNA can be recovered by collecting only the upper layer. By adding a lower alcohol, such as isopropanol, to the collected solution to precipitate RNA, after washing, RNA of high purity can be obtained. The extracted RNA can be used as total RNA or as purified mRNA.

Various methods can be employed to determine the amount of the extracted RNA. For example, RT-PCR, real-time RT-PCR, invader assay, DNA chip, Northern blot analysis, etc., can be employed.

The RT-PCR and real-time RT-PCR are methods in which complementary DNA (cDNA) is synthesized from mRNA, and DNA in the object region is synthesized using a suitable primer and DNA polymerase (generally, heat resistant DNA polymerase). Generally, the amount of RNA is measured after amplifying DNA by repeating denaturation, annealing and elongation of DNA.

The primers used in RT-PCR or real-time RT-PCR may use any base sequence region, as long as they comprise regions that can specifically amplify the object genes. The length of the base sequence is not particularly limited. Generally, base sequences having a length of from 20 to 30 nucleotides are used.

CYBR Green, PicoGreen, ethidium bromide, etc., are used as fluorescent molecules having affinity with DNA strands used in real-time RT-PCR. CYBR Green is preferably used.

RT-PCR and real-time RT-PCR are preferably employed because they can amplify DNA to several 100,000 times, are highly sensitive, and only a small amount of test sample is required.

The invader assay is a method comprising the steps of: on RNA or DNA, by linking a probe (invader probe) that is complementary to the RNA or DNA and a complementary probe (signal probe) that has a noncomplementary region at the 5′-end, cutting the signal probe by a Cleavase enzyme that recognizes the conformation; and measuring the expression levels of genes by detecting any fragments of the cut off signal probe.

As an invader probe, it is possible to use a probe that is homologous to the object transcript and there is no particular limitation to its base sequence length. There is no limitation to the length, etc. of the base sequence of the signal probe, as long as it has a base sequence that forms a triple strand structure with the invader probe on the transcript; specifically, a probe that has a noncomplementary region at the 5′-end and a complementary region at the 3′-end, and Cleavase can recognize the conformation and cut the signal probe off. The method for measuring or detecting the cut off fragment of the signal probe is such that, for example, when the fragment of the signal probe is a noncomplementary region of the transcript, the fragment with the fluorescent labeled probe is cut off by Cleavase and the fluorescent signal of the obtained fragment is measured. There is no limitation to the fluorescent labeled probe used in this case as long as it is a DNA probe having a base sequence complementary in a portion to the fragment of the signal probe, and, when the fragment is hybridized, a conformation recognizable by Cleavase as described above is formed, wherein a luminous material labels the cut off portion and a quenching material labels the uncut portion, and luminous signals are not emitted when it is in an uncut condition. As the luminous material, generally a fluorescent material, a phosphorescent material, etc., are preferably used. As the quenching substance, Cy3, etc., are preferably used.

When the signal probe fragment has a region that is complementary to the transcript, for example, it is possible to exemplify the method such that an immobilized oligonucleotide having a region complementary to the fragment is made to bind the separated fragment, and the fragment is detected by a fluorescence antibody method using fluorescein as a fluorescent pigment. This can be conducted using commercially available measurement kits, etc.

The invader assay is preferably employed because the probe itself does not have to be labeled and an amplification operation is unnecessary.

A DNA chip is explained in detail in the section entitled (5) DNA chip.

The method for determining the amount of translated products of gene is performed by quantifying the protein coded by the object gene. Specifically, it is possible to exemplify a method such that the amount of protein coded by the object gene is determined by employing an immunoassay using an antibody that can specifically recognize proteins. As the immunoassay, Western blot analysis, radioimmunoassay, ELISA, etc., can be exemplified.

In the detection method of the present invention, expression levels of genes in the tested tissue measured by the above-descried methods are compared to those of the corresponding genes in a control.

Specifically, (a), in the tested tissue, the expression level(s) of at least one gene selected from the group consisting of plasminogen gene, EST51549, retinol-binding protein 4 gene and organic anion transporter C gene is measured; and

(b) the expression level(s) of the genes measured in (a) is compared to the expression levels of the genes in the control that correspond to the gene(s) measured in (a).

Alternatively, (a), in the tested tissue, the expression level(s) of at least one gene selected from the group consisting of plasminogen gene, EST51549, retinol-binding protein 4 gene and organic anion transporter C gene and the expression level(s) of at least one gene selected from the group consisting of aldolase B gene, carbamyl phosphate synthase 1 gene, albumin gene, cytochrome P450 subfamily 2E1 gene are measured, and

(b) the expression levels of the genes measured in (a) are compared to the expression levels of the genes in the control that correspond to the genes measured in (a).

The tissue used as the control can be suitably selected depending on the means employed in the detection method or the purpose of detection. Specifically, tissue from nonpatients, noncancerous regions of hepatic tissue of chronic hepatitis patients, human peripheral blood mononuclear cells of nonpatients, etc., can be used.

When the expression levels of genes in the tested tissues are lower than the expression levels of corresponding genes in the control, it is assessed that the tested tissue is of hepatocellular carcinoma or the tested tissue includes hepatocellular carcinoma, and therefore hepatocellular carcinoma can be detected.

(3) Process for the Judgment

By measuring the expression levels of the above-mentioned 8 genes, it is possible to determine if a tested tissue has hepatocellular carcinoma and to assess the malignancy of the hepatocellular carcinoma, etc.

Specifically, it can be conducted by following the procedure as below:

First, in the tested tissue, the expression level(s) of at least one gene selected from the group consisting of plasminogen gene, EST51549, retinol-binding protein 4 gene and organic anion transporter C gene is measured.

Alternatively, in the tested tissue, the expression level(s) of at least one gene selected from the group consisting of plasminogen gene, EST51549, retinol-binding protein 4 gene and organic anion transporter C gene and the expression level(s) of at least one gene selected from the group consisting of aldolase B gene, carbamyl phosphate synthase 1 gene, albumin gene and cytochrome P450 subfamily 2E1 gene are measured.

The expression levels of the genes in the tested tissue are then compared to the expression levels of corresponding genes in the control.

When the expression levels of genes of the tested tissue are lower than those of the genes in the control, it is assumed that the tested tissue has hepatocellular carcinoma or there is a high possibility that hepatocellular carcinoma or like cancer cell is included in the tested tissue. It is also possible to assess the malignancy of the cancer based on the degree of underexpression.

This assessment method can be utilized in diagnosis or treatment of hepatitis patients.

(4) Early Detection Method

By periodically measuring the expression levels of the 8 genes, early detection of hepatocellular carcinoma or a region having a high possibility of developing into hepatocellular carcinoma becomes possible.

Specifically, it can be conducted by following the procedure as below:

First, in the tested tissue, the expression level(s) of at least one gene selected from the group consisting of plasminogen gene, EST51549, retinol-binding protein 4 gene and organic anion transporter C gene is periodically measured.

Alternatively, in the tested tissue, the expression level(s) of at least one gene selected from the group consisting of plasminogen gene, EST51549, retinol-binding protein 4 gene and organic anion transporter C gene and the expression level(s) of at least one gene selected from the group consisting of aldolase B gene, carbamyl phosphate synthase 1 gene, albumin gene and cytochrome P450 subfamily 2E1 gene are periodically measured.

As a result of the periodic measurement, when underexpression compared to a previously measured expression level is observed, it is possible to assess the tested tissue as having hepatocellular carcinoma or having a region highly possible to develop to hepatocellular carcinoma.

By periodically measuring the change in the expression levels of the specific genes, it is possible to detect occurrence or development of hepatocellular carcinoma at an early stage.

The measuring period, i.e., the duration of the period between measurements, can be suitably selected depending on the condition of the patient or tested individual. For example, periodic measurement can be performed once per a half-year or once per a year.

The early detection of the present invention can be used for preventing or treating hepatocellular carcinoma, or analysis for prognosis of a hepatitis patient.

(5) DNA Chip

The detection method, process for the judgment, and early detection method can effectively conducted by using a DNA chip in which whole or a part of DNA comprising transcribed region(s) of the gene(s) to be measured, i.e., at least one gene in the tested tissue selected from the group consisting of plasminogen gene, EST51549, retinol-binding protein 4 gene and organic anion transporter C gene, is immobilized. Furthermore, the method can be more effectively conducted by using a DNA chip in which whole or a part of DNA comprising transcribed regions of, in addition to the above-mentioned genes, at least one gene in the tested tissue selected from the group consisting of aldolase B gene, carbamyl phosphate synthase 1 gene, albumin gene and cytochrome P450 subfamily 2E1 gene, is immobilized.

Among those types in which DNAs of a gene are immobilized on a surface, some are categorized as DNA arrays. The DNA arrays can be grouped into DNA microarrays and DNA macroarrays. The DNA chip of the present invention includes these so-called DNA arrays (including DNA microarrays and DNA macroarrays).

The DNA chip of the present invention can be produced by synthesizing whole or a part of the DNA comprising the transcribed regions of the genes to be measured by employing a conventional method, and immobilizing the DNA on a support or directly synthesizing it on a support).

There is no limitation to the support (or the surface) as long as it can immobilize DNA. For example, a silicon chip, a glass slide, a nylon membrane, etc., can be used.

There is no limitation to the immobilization method as long as it is a generally used method. Methods in which DNA is spotted using a spotter, an arrayer, etc., or in which synthesis of nucleotides is sequentially performed on a support, etc., are preferably employed.

There is no limitation to the region and the length of the base sequence of the DNA immobilized on the support as long as it specifically hybridizes with a labeled cDNA prepared from transcripts of the above-mentioned 8 genes.

For example, PCR products based on cDNA prepared from the transcripts of the 8 genes, synthesized oligonucleotides or their partial fragments prepared in accordance with the base sequences of the transcribed regions of the 8 genes, etc., can be preferably used.

The DNA chip of the invention is, specifically, produced by immobilizing whole or a part of the DNA comprising a transcribed region of at least one gene selected from the group consisting of plasminogen gene, EST51549, retinol-binding protein 4 gene and organic anion transporter C gene.

The DNA chip of the invention can also be produced by immobilizing whole or a part of the DNA, in the tested tissue comprising transcribed regions of at least one gene selected from the group consisting of plasminogen gene, EST51549, retinol-binding protein 4 gene and organic anion transporter C gene, and, at least one gene selected from the group consisting of aldolase B gene, carbamyl phosphate synthase 1 gene, albumin gene and cytochrome P450 subfamily 2E1 gene.

In the DNA chip of the present invention, it is possible to further immobilize, if desired, whole or a part of DNA comprising transcribed region(s) of known genes other than the above 8 genes, synthesized nucleotides or their fragments, etc.

The DNA chip of the present invention can be used for detecting. hepatocellular carcinoma. To be more specific, it can be used for diagnosing hepatocellular carcinoma or detecting hepatocellular carcinoma at an early stage.

Specifically, the DNA chip of the invention can be used in the following manner:

From the tested tissue, the transcripts of at least one gene selected from the group consisting of plasminogen gene, EST51549, retinol-binding protein 4 gene and organic anion transporter C gene, and at least one gene selected from the group consisting of aldolase B gene, carbamyl phosphate synthase 1 gene, albumin gene and cytochrome P450 subfamily 2E1 gene are extracted. By hybridizing the labeled cDNA prepared from the transcripts of the genes on the DNA chip of the invention, the expression levels of the genes can be measured. The measurement for detecting hepatocellular carcinoma according to the invention can be thereby performed.

cDNAs prepared from the transcripts of the tested tissue and the control are each labeled by different colorants, such as Fluorescein (green), Phycoerythrin (red), a substance having biotin added to Fluorescein or Phycoerythrin, Cy3-deoxyuridine triphosphate, dUTP, and Cy5-dUTP. The cDNAs are linked to the DNA chip and the difference in the intensity of fluorescence is processed by computer. By numerically expressing the degree of underexpression of the targeted genes in the tested tissue compared to those in the control, the method for detecting hepatoma cells of the present invention can be conducted. During this step, it is also possible to detect hepatoma cells by visually identifying the differences in color using a fluorescence microscope.

The operations employed in the detection method, process for the judgment, early detection method of the invention, or production of the DNA chip, for example, chemical synthesis, cutting, removing, linking or adding of DNA, and isolation, purification, amplification or reproduction of enzymes used for synthesizing cDNA of gene transcripts or transcripts of genes, etc., can be conducted by methods that were known before the filing date of the present application. Determination or confirmation of base sequences can be performed by, for example, the dideoxy method or Maxam-Gilbert method.

### EXAMPLES

The present invention is explained below in further detail with reference to Examples. However, the scope of the invention is not limited to these Examples.

1. Determination of a gene that is underexpressed in hepatocellular carcinoma.

Cells in a hepatocellular carcinom lesion of the liver tissue of chronic viral hepatitis patients (two patients infected with HBV and two patients with HCV) and cells in a noncancerous region of the same liver tissue were used as samples. Total RNAs were extracted from the cancerous tissue and noncancerous tissue surgically resected from hepatitis patients. To 1 μg of total RNAs, ROX-fluorescent-labeled 3′-anchored oligo-dT (oligo-dT; GT15MG, GT15MA, GT15MT, GT15MC, where M represents a mixture of G, A and C, synthesized by Greiner Labortechnik Japan/Japan, 50 pmol in 11 μl of diethylpyrocarbonate-treated water) was added and heated at 70° C. for 10 minutes. Solution A having the following composition was then added thereto to obtain a final volume of 20 μl.

(Composition of Solution A)

4 μl of 5 × first-strand buffer (0.25 M tris-HCl, pH7.5; 0.375 mol/L KCl; 0.05 mol/L dithiothreitol and 0.015 mol/L MgCl₂) 2 μl of 0.1 mol/L dithiothreitol, 1 μl of 2.5 mmol/l deoxynucleotide triphosphates (dNTPs), 1 μl of ribonuclease inhibitor (40 units; Wako Pure Chemical Industries, Japan) and 1 μl of superscript II reverse transcriptase (200 units; BRL, USA).

The RNA solution was incubated at 42° C. for one hour to synthesize cDNA and then diluted 5-fold by the addition of 80 μl of diethylpyrocarbonate-treated water. Using the resulting cDNA as a temlate, amplification of the object genes was conducted by PCR. The added reagents and the reaction conditions were as follows:

Added reagents: 2 μl of reaction solution, 2 μl of 10×PCR buffer (100 mmol/L Tris-HCl, 15 mmol/L MgCl₂, 500 mmol/L KCl and 1 mg/ml geratin, pH8.5), 1.6 μl of 2.5 mmol/L dNTPs, 0.2 μl of Taq DNA polymerase (5 units/μl; Roche Molecular Systems, NJ), 5 pmol of ROX-fluorescent-labeled 3′-anchored oligo-dT primer and 10 pmol of ROX-fluorescent-labeled 5′-anchored oligo-dT primer.

Reaction conditions: one cycle of 3 minutes at 95° C., 5 minutes at 40° C., and 5 minutes at 72 C.; then 2 to 40 cycles of 30 seconds at 95° C., 2 minutes at 40° C., and 5 minutes at 72° C.

Each reaction solution prepared from the cancerous tissue and noncancerous tissue as described above was electrophoresed on 6% polyacrylamide gel containing 7.5 M urea. Using an FM BIO II imaging analyzer (Takara Holdings Inc.), the expression levels of genes were analyzed. As a result, it was found that there was a difference in intensity of fluorescence between the genes in noncancerous tissue and cancerous tissue and a plurality of genes (shown by arrows) apparently underexpressed in the cancerous tissue (FIG. 1). The bands showing differences in intensity of fluorescence were cut and the fragments were immersed in a 100 μl of TE (Tris-HCl, EDTA) buffer for one hour and DNAs were extracted. Thereafter, reamplification was conducted by PCR using the extract solution as a template. The reaction conditions were the same as those in the first PCR.

The reamplified PCR products were electrophoresed on 3% agarose gel, the bands thereof were cut, and recovered using GFX PCR DNA and Gel Band Purification Kit (Amersham Pharmacia Biotech, NJ). The DNAs of the reamplified PCR products were cloned using the cloning vector pCRII (Invitrogen Japan, Japan), and strands of DNAs were sequenced using an ABI377 (Applied Biosystems, USA).

As the result of the above operation, 8 genes that show significantly decreased expression levels were identified.

The nucleotide sequences of the 8 genes were analyzed using the GenBank database and it was determined that these 8 genes had the nucleotide sequences of Seq. Nos. 1 to 8. In other words, they are 8 genes, namely aldolase B gene, carbamyl phosphate synthase 1 gene, plasminogen gene, EST51549, albumin gene, cytochrome P450 subfamily 2E1 gene, retinol-binding protein 4 gene and organic anion transporter C gene.

2. Measurement of expression levels of genes in a cancerous cell underexpressed in hepatocellular carcinoma Real-time RT-PCR was conducted based on total RNAs prepared by extracting from chronic hepatitis patients derived hepatocellular carcinoma tissue (samples surgically obtained from 20 patients each with chronic hepatitis).

Specifically, amplification reaction was performed using a 20 μl of total RNA solution containing 2 μl of 10× reaction buffer (Taq polymerase, dNTP, MgCl₂ and CYBR Green fluorescent (Roche Diagnostics) and 2 μl of template cDNA with each oligonucleotide primer.

Reaction conditions: 40 cycles of 10 seconds at 95° C., 10 seconds at 65° C. and 30 seconds at 72° C. As a PCR amplifier, Light Cycler (Roche Diagnostics, Germany) was used.

The measured expressions were compared to those of the noncancerous tissue of the same chronic hepatitis patient. Table 1 shows the results.

TABLE 1

| | | Hepatocellular carcinoma patients with underexpression over 50% | | | |
|---|---|---|---|---|---|
| | GenBank | HBV (−), HCV (−) | HBV (+), HCV (−) | HBV (−), HCV (+) | total |
| Genes | ACC. No. | (n = 2) | (n = 3) | (n = 15) | (n = 20) |
| Aldolase B gene | X02747 | 2/2 (100%) | 2/3 (66.7%) | 14/15 (93.3%) | 18/20 (90.0%) |
| Carbamyl phosphate synthase | D90282 | 2/2 (100%) | 2/3 (66.7%) | 11/15 (73.3%) | 15/20 (75.0%) |
| I gene | | | | | |
| Phasminogen gene | X05199 | 2/2 (100%) | 2/3 (66.7%) | 11/15 (73.3%) | 15/20 (75.0%) |
| EST51549 | AA345522 | 2/2 (100%) | 2/3 (66.7%) | 11/15 (73.3%) | 15/20 (75.0%) |
| Albumin gene | V00495 | 2/2 (100%) | 1/3 (33.3%) | 12/15 (80.0%) | 15/20 (75.0%) |
| Cytochrome P450 subfamily | J02843 | 2/2 (100%) | 1/3 (33.3%) | 10/15 (66.7%) | 13/20 (65.0%) |
| 2E1 gene | | | | | |
| Retinol-binding protein 4 gene | X00129 | 2/2 (100%) | 1/3 (33.3%) | 9/15 (60.0%) | 12/20 (60.0%) |
| Organic anion transporter | AB026257 | 2/2 (100%) | 1/3 (33.3%) | 8/15 (53.3%) | 11/20 (55.0%) |
| C gene | | | | | |

In table 1, HBV indicates hepatitis B virus and HCV indicates hepatitis C virus. (+) indicates that the patient was infected by the virus and (−) indicates that the patient was not infected by the virus.

In the fractions of table 1, the numerators express the number of patients with underexpression over 50% and the denominators express the total number of tested patients. The percentage numbers in ( ) indicate the percentage ratio of the number of patients with underexpression over 50% to the total number of the tested patients.

As shown in table 1, regardless of the type of chronic hepatitis, patients with hepatocellular carcinoma showed a significant decrease, i.e., over 50%, in the expression levels of the 8 genes.

As described above, aldolase B gene, carbamyl phosphate synthase 1 gene, plasminogen gene, EST51549, albumin gene, cytochrome P450 subfamily 2E1 gene, retinol-binding protein 4 gene and organic anion transporter C gene are underexpressed in hepatocellular carcinoma tissue.

By measuring the expressions of these genes and determining if the expressions thereof are decreased compared to those of a control, it becomes possible to accurately detect hepatocellular carcinoma. Furthermore, by measuring the expressions of the genes or degree of underexpression thereof, if decreased, diagnosis of hepatocellular carcinoma or early detection thereof can be properly performed. The DNA chip obtained by immobilizing whole or a part of the DNA comprising the transcribed regions of the 8 genes can be used as an effective tool for detecting hepatocellular carcinoma.

As described above, the technique of the present invention can be effectively used for prevention, diagnosis, or treatment of hepatocellular carcinoma, and analysis for prognosis of chronic hepatitis, etc.

1. A method for detecting hepatocellular carcinoma comprising the step of:
(a) measuring, in a tested tissue, the expression level(s) of at least one gene selected from the group consisting of plasminogen gene, EST51549, retinol-binding protein 4 gene and organic anion transporter C gene; and
(b) comparing the expression level(s) of the gene(s) measured in (a) with the expression levels of the genes in a control that correspond to the genes measured on step (a).

2. A method for detecting hepatocellular carcinoma comprising the step of:
(a) measuring, in a tested tissue, the expression level(s) of at least one gene selected from the group consisting of plasminogen gene, EST1549, retinol-binding protein 4 gene and organic anion transporter C gene, and at least one gene selected from the group consisting of aldolase B gene, carbamyl phosphate synthase 1 gene, albumin gene and cytochrome P450 subfamily 2E1 gene; and
(b) comparing the expression levels of genes measured in (a) with the expression levels of genes in a control that correspond to the genes measured in (a).

3. A method for detecting hepatocellular carcinoma according to claim 1, wherein the step (a) of measuring the expression level(s) of the gene(s) is performed by determining the amount of transcripts of the genes being measured.

4. A method for detecting hepatocellular carcinoma according to claim 1, wherein the step (a) of measuring the expression level(s) of the gene(s) is performed by amplifying whole or a part of the DNA to be measured and using cDNA prepared from gene transcripts as a template.

5. A method for detecting hepatocellular carcinoma according to claim 1, wherein the step (a) of measuring the expression level(s) of the gene(s) is performed by invader assay.

6. A method for detecting hepatocellular carcinoma according to claim 1, wherein the step (a) of measuring the expression level(s) of the gene(s) is performed by hybridizing labeled cDNA prepared from transcripts including the gene(s) to be measured with whole or a part of the immobilized DNA of the gene(s) to be measured.

7. A method for detecting hepatocellular carcinoma according to claim 1, wherein the tested tissue in the step (a) is liver tissue of a chronic hepatitis patient.

8. A method for detecting hepatocellular carcinoma at an early stage that comprises the step of periodically measuring the expression level(s), in a tested tissue, of at least one gene selected from the group consisting of plasminogen gene, EST51549, retinol-binding protein 4 gene and organic anion transporter C gene.

9. A method for detecting hepatocellular carcinoma at an early stage that comprises the step of periodically measuring the expression level(s), in a tested tissue, of at least one gene selected from the group consisting of plasminogen gene, EST51549, retinol-binding protein 4 gene and organic anion transporter C gene, and at least one gene selected from the group consisting of aldolase B gene, carbamyl phosphate synthase 1 gene, albumin gene and cytochrome P450 subfamily 2E1 gene.

10. A DNA chip for detecting hepatocellular carcinoma in which whole or a part of DNA comprising transcribed region(s) of at least one gene selected from the group consisting of plasminogen gene, EST51549, retinol-binding protein 4 gene and organic anion transporter C gene is immobilized.

11. A DNA chip for detecting hepatocellular carcinoma in which whole or a part of DNA, in a tested tissue, comprising transcribed region(s) of at least one gene selected from the group consisting of plasminogen gene, EST51549, retinol-binding protein 4 gene and organic anion transporter C gene, and, at least one gene selected from the group consisting of aldolase B gene, carbamyl phosphate synthase 1 gene, albumin gene and cytochrome P450 subfamily 2E1 gene.
